(19) United States
(12) Patent Application Publication
(10) Pub. No.: US 2023/0065466 A1
(43) Pub. Date: Mar. 2, 2023

(54) METHODS AND SYSTEMS FOR MOBILE COMMUNICATIONS

(71) Applicant: UNITED STATES GOVERNMENT AS REPRESENTED BY THE DEPARTMENT OF VETERANS AFFAIRS, Washington, DC (US)

(72) Inventors: Deanna Callahan, Loma Linda, CA (US); Shane M Elliot, Loma Linda, CA (US); Robert Durkin, Washington, DC (US)

(21) Appl. No.: 17/766,415

(22) PCT Filed: Oct. 5, 2020
(86) PCT No.: PCT/US2020/054280

§ 371 Date: Apr. 4, 2022

Related U.S. Application Data
(60) Provisional application No. 62/910,202, filed on Oct. 3, 2019.

(51) Int. Cl. G16H 40/20; G06Q 10/10; G16H 10/60

(52) U.S. Cl. CPC G16H 40/20; G06Q 10/1095; G16H 10/60

(57) ABSTRACT

A method may comprise determining, based on a date, a patient identified for a patient scheduled for an appointment, determining, based on the patient identifier, a mobile device number associated with the patient, sending, based on the mobile device number, an interactive message, and receiving, in response to the interactive message, an indication that the appointment should be maintained or canceled.

### CROSS-REFERENCE TO RELATED APPLICATION

This application claims the benefit of U.S. Provisional Application No. 62/910,202, filed Oct. 3, 2019, which is hereby incorporated by reference in its entirety.

### BACKGROUND

The use of text messages (e.g., Short Message Service (SMS)) to remind patients of their upcoming appointment addresses clinical care issues. When a patient does not come to their scheduled medical appointment, the provider and ancillary support clinical staffs time set aside for the patient's appointment is not used resulting in inefficiencies and an appointment time that could have been given to another patient. Currently, text message reminders to patients are limited because of requirements imposed by various privacy guidelines.

### SUMMARY

In an aspect, described are methods for facilitating patent visits. A method may comprise determining, based on a date, a patient identifier for a patient scheduled for an appointment, determining, based on the patient identifier, a mobile device number associated with the patient, sending, based on the mobile device number, an interactive message, and receiving, in response to the interactive message, an indication that the appointment should be maintained or canceled.

In an aspect, described are methods comprising receiving, in response to a first interactive message, an indication that an appointment should be canceled, determining, based on the appointment, an associated clinic, determining, based on the associated clinic and a date associated with the canceled appointment, a patient identifier in a waiting list for an appointment with the clinic, wherein the patient identifier has been in the waiting list for greater than a threshold period of time, determining, based on the patient identifier, a mobile device number, sending, based on the mobile device number, a second interactive message offering an appointment at the associated clinic on the date of the canceled appointment, and receiving, in response to the second interactive message, an indication that the appointment should be established.

In an aspect, described are methods for facilitating patient check-in comprising receiving an indication that a patient has arrived at a location, sending, based on the indication, an interactive message, receiving a response to the interactive message, and performing, based on the response, a patient check-in. The interactive message may comprise a questionnaire regarding current health conditions (e.g., symptoms).

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

To easily identify the discussion of any particular element or act, the most significant digit or digits in a reference number refer to the figure number in which that element is first introduced.

FIG. 1 illustrates an example system;

FIG. 2 illustrates an example scheduling engine;

FIG. 3 illustrates an example data snapshot;

FIG. 4A illustrates an example messaging interface;

FIG. 4B illustrates an example messaging interface;

FIG. 4C illustrates an example messaging interface;

FIG. 4D illustrates an example messaging interface;

FIG. 5 illustrates an example method;

FIG. 6 illustrates an example method; and

FIG. 7 illustrates an example system.

### DETAILED DESCRIPTION

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes—from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the examples included therein and to the Figures and their previous and following description.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

In an aspect, described are methods for facilitating patent visits. The method may comprise determining a patient identifier. The patient identifier may be stored in a database. The patient identifier may be associated with one or more dates. For example, the one or more dates may comprise scheduled appointment dates. The patient identifier may further be associated with a patient contact. The patient contact may comprise, for example, a phone number or email address. The patient contact may be determined based on the patient identifier. A message may be sent to the patient. For example, if the patient contact comprises a mobile number, the message may comprise a text message (e.g., SMS message). The message may comprise an interactive element and/or instructions for reply. For example, the message may comprise an instruction to click a hyperlink (e.g., the interactive element) to confirm the scheduled appointment. For example, the message may comprise an instruction to text a particular word (e.g., “confirm”) to a particular number. Based on the message, an indication may be received. For example, the indication may indicate that the patient has confirmed the appointment, canceled the appointment, or rescheduled the appointment.

In an aspect, if the indication indicates the appointment should be canceled, the method may further comprise determining, based on the appointment, an associated clinic. For example, the patient identifier and/or the one or more dates may be associated with the associated clinic. For example, the scheduled appointment may comprise an appointment at a vision clinic. The method may further comprise, determining, based on the associated clinic and a date associated with the canceled appointment, a second patient identifier in a waiting list for an appointment with the clinic, wherein the second patient identifier has been in the waiting list for greater than a threshold period of time. The second patient identifier may be associated with another patient who also has an appointment at the associated clinic. The method may further comprise, determining, based on the second patient identifier, a second mobile device number, sending, based on the second mobile device number, a second interactive message offering an appointment at the associated clinic on the date of the canceled appointment, and receiving, in response to the second interactive message, a second indication that the appointment should be established. For example, a first patient may indicate that the first patient would like to cancel the appointment and thus, the appointment slot should be offered to another patient. Thus, a message may be sent to the another patient informing the another patient of the open slot and may present an opportunity to the another patient to fill the open slot (e.g., schedule an appointment).

In an aspect, if the indication indicates the scheduled appointment is confirmed, upon arrival at the associated clinic (or before or after or any other time relative to the scheduled appointment) the patient may send a message indicating the patient's presence in the vicinity of the associated clinic (e.g., an “I Am Here” message). The message indicating the patient's presence in the vicinity of the associated clinic may be received. In response to receiving the message indicating the patient's presence in the vicinity of the associated clinic, an interactive message may be sent. For example, the interactive message may be sent to the client contact. The interactive message may comprise instructions. For example, the interactive message may comprise instructions associated with parking, entry/exit locations, and the like. Further, the interactive message may comprise an interactive element. The interactive element may be configured to receive a patient input. For example, the interactive element may display a question and provide a hyperlink or other element to receive a response. For example, the interactive element may ask about, and receive inputs regarding, patient symptoms. For example, the interactive element may as “do you have a fever?” or “have you been exposed to COVID-19?” or any other question. The patient may input a patient input (e.g., a response to a question). The patient input may be relayed to the associated clinic as described further herein. Upon receipt of the patient input, the patient may be “checked-in” to their appointment. A patient may be checked-in to their appointment when the provider is available and ready to see the patient, and likewise the patient is available and ready to be seen (e.g., the patient is at the clinic). Receiving an “I Am Here” message or sending a check-in message may cause the health information system and scheduling engine to update the status of the appointment. For example, the system may change the status to “checked-in” after sending the check-in message. After the appointment is completed (e.g., the physician has seen the patient and the patient has been discharged), the status may be changed to “checked-out.”

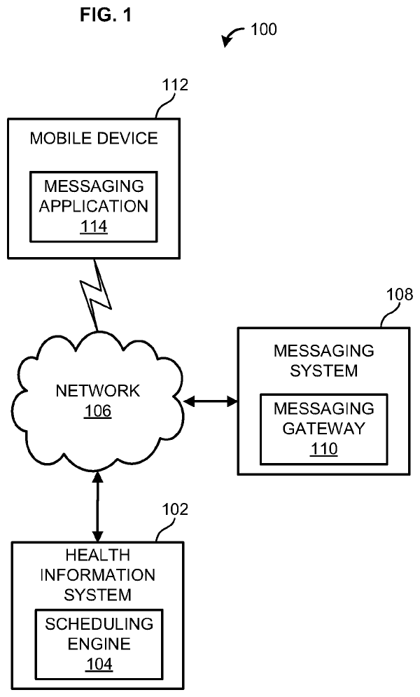

FIG. 1 is a block diagram depicting non-limiting examples of a system 100 comprising a health information system 102, a messaging system 108, and one or more mobile devices 112 connected through a network 106. The health information system 102 can comprise one or more computers, such as, for example, a laptop computer, a desktop computer, a mobile phone (e.g., smartphone), a tablet, combinations thereof, and the like. The health information system 102 can be configured to operate a scheduling engine 104. The messaging system 108 can comprise one or multiple computers, such as, for example, a laptop computer, a desktop computer, a mobile phone (e.g., smartphone), a tablet, combinations thereof, and the like. The messaging system 108 can be configured to operate a messaging gateway 110. The mobile device 112 can be a computer, such as, for example, a laptop computer, a desktop computer, a mobile phone (e.g., smartphone), a tablet, and the like. The mobile device 112 can be configured to operate a messaging application 114. The mobile device(s) 112 can be in communication with the health information system 102 and/or the messaging system 108 through the network 106. The network 106 may be configured for communicatively coupling the various components of the system 100. In one embodiment, the network 106 may be wired or wireless, and may have numerous different configurations. Furthermore, the network 106 may include a local area network (LAN), a wide area network (WAN) (e.g., the Internet), and/or other interconnected data paths across which multiple devices (e.g., the health information system 102, the messaging system 108, the mobile device(s) 112, etc.) may communicate. In some embodiments, the network 106 may be a peer-to-peer network. The network 106 may also be coupled with portions of a telecommunications network for sending data using one or more communication protocols. In some embodiments, the network 106 may include a Bluetooth (or Bluetooth low energy) communication network, a cellular communications network for sending and receiving data including via short messaging service (SMS), multimedia messaging service (MMS), hypertext transfer protocol (HTTP), direct data connection, wireless access protocol (WAP), email, etc.

The health information system 102 can be configured to operate the scheduling engine 104 which can be configured for scheduling appointments for one or more patients, such as confirming appointments, canceling appointments, rescheduling appointments, combinations thereof, and the like. The appointments may be any type of appointment, including but not limited to, medical appointments, dental appointments, legal appointments, vehicle service appointments, veterinary appointments, and the like. The health information system 102 can be further configured to comprise, and/or communicate with, an electronic medical records component.

The messaging system 108 may comprise a web-based server infrastructure to operate the messaging gateway 110 which is configured for sending messages, such as SMS (short message service) or text messages, to the mobile device(s) 112. An application programming interface (API) can enable the scheduling engine 104 of the health information system 102 to send and receive messages via the messaging system 108. As used herein, the term API should be understood to mean any combination of software, firmware, and/or hardware that allows two or more software applications (e.g., machine-readable instructions) to communicate with one another. The scheduling engine 104 may communicate with the messaging gateway 110 via an HTTP SSL connection to securely send and receive messages. The messaging gateway 110 may be Short Message Service (SMS) gateway configured for receiving and sending SMS transmissions to and/or from components coupled to the network 106. In essence, the messaging system 108 may, for example, receive and/or route messages or messaging opportunities via the network 106 between the mobile device(s) 112 and the health information system 102. While the messaging system 108 may be described in the context of SMS transmissions, it should be understood that the messaging system 108 may also be configured to operate using other message transmissions such as email, mobile push messaging, embedded messaging, other third-party messaging services, etc.

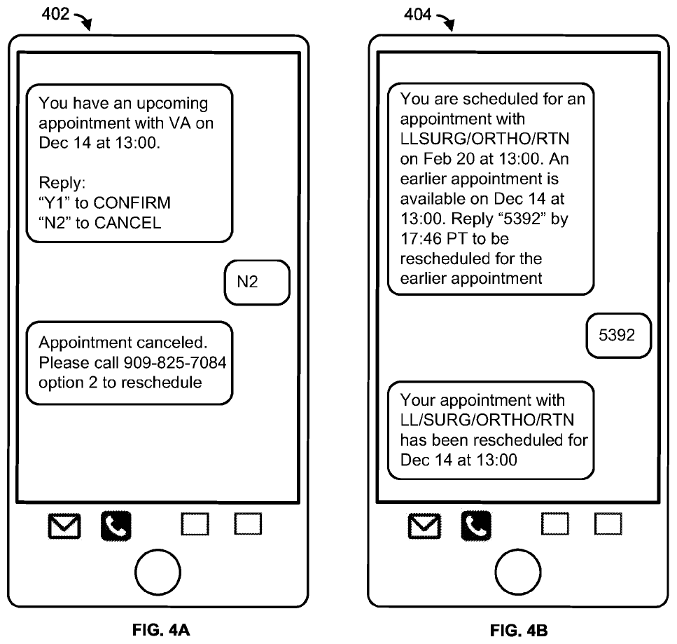

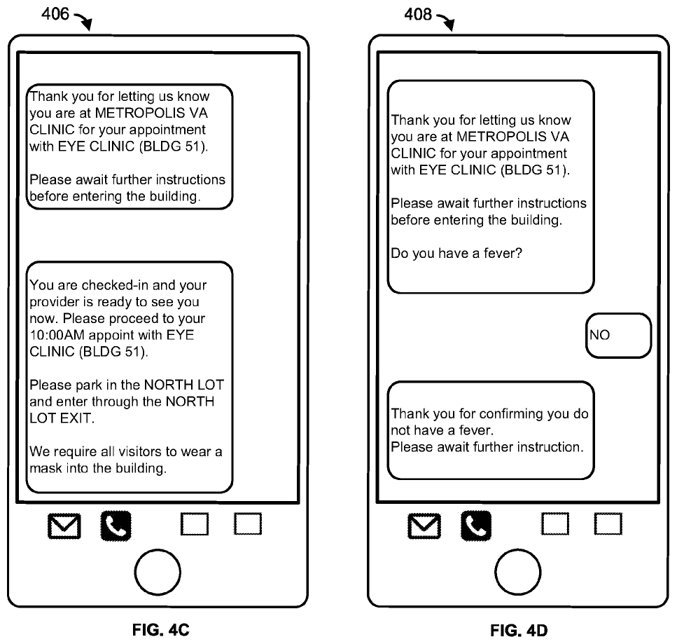

The mobile device(s) 112 may be configured to operate the messaging application 114, which in turn may be configured to receive secure and/or unsecure messages via the network 106, receive one or more inputs from a user, and send a response via the network 106 to the health information system 102, the messaging system 108, and/or other components of the system 100. The scheduling engine 104 may be communicatively coupled with via the network 106 to either or both of the messaging system 108 (and the messaging gateway 110), and the mobile device 112 (and the messaging application 114). The scheduling engine may be configured to send messages (e.g., data) to either or both of the mobile device 112 and the messaging system 108. For example, the scheduling engine may interface with the messaging system 108 via the messaging gateway 110 so as to generate a message to be sent to the mobile device 112. For example, the scheduling engine may query data from a database (e.g., patient information such as a patient ID, a patient contact, a scheduled appoint, combinations thereof, and the like). The scheduling engine 104 may send the patient information to the messaging system 108. The messaging system 108 may receive the patient information and generate a message. The messaging system 108 may send the message. For example, the message may be sent to a patient contact stored in the database. The message may comprise additional patient information such as a patient name and a scheduled appointment time. The message may comprise an interactive element and/or instructions (as explained further herein with reference to FIGS. 4A-4D). Via the interactive element and/or in accordance with the instructions, the messaging system 108 may receive a response sent from the mobile device 112. For example, the response may indicate that a scheduled appointment is to be maintained, canceled, or rescheduled. Based on receiving the response indicating the scheduled appointment is to be maintained, canceled, or rescheduled, the messaging system may instruct the health information to update the appointment (e.g., with an indicator that the appointment is confirmed, canceled, or rescheduled). Based on determining that the scheduled appointment is canceled the health information system may determine that the time associated with the scheduled appointment is now an “open slot.” Based on determining the open slot, the health information system may determine a second patient ID associated with a clinic associated with the original scheduled appointment. If a second patient ID associated with the clinic associated with the original scheduled appointment is identified, the health information system 102 may send the second patient ID to the messaging system 108 with instructions to generate and send a message to a patient contact associated with the second patient ID offering the scheduled appointment to the second patient.

If the original appointment is confirmed or the open slot is filled, the messaging system, at or around the time of the appointment, may receive a message from the mobile device 112 indicating the patient has arrived (e.g., an “I Am Here” message). The I Am Here message may indicate the patient associated with the scheduled appointment is in the vicinity of the location at which the appointment is to take place (e.g., a clinic associated with the appointment). Based on the I Am Here message, the health information system may “check-in” the patient for the scheduled appointment. In response to receiving the I Am Here message, the messaging system 108 may send an instructional message to the mobile device 112. For example, the instructional message may comprise an interactive element and/or instructions. For example, the instructional message may instruct the patient to park in a certain parking lot, to enter a building via a certain entrance, to wear a mask inside a building, to complete a questionnaire (e.g., a health screening form), combinations thereof, and the like. A user may, based on the instructional message, complete the questionnaire, thereby generating a health screening message which may be sent to the health information system 102.

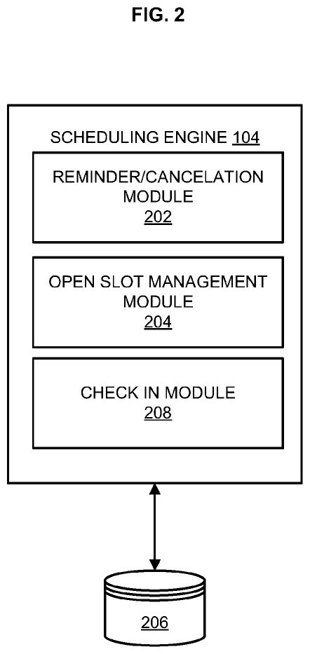

FIG. 2 is a block diagram depicting an example view of the scheduling engine 104 in communication with a scheduling database 206. The scheduling engine 104 can comprise one or more of, a reminder/cancellation module 202, an open slot management (OMS) module 204, and a check in module 208.

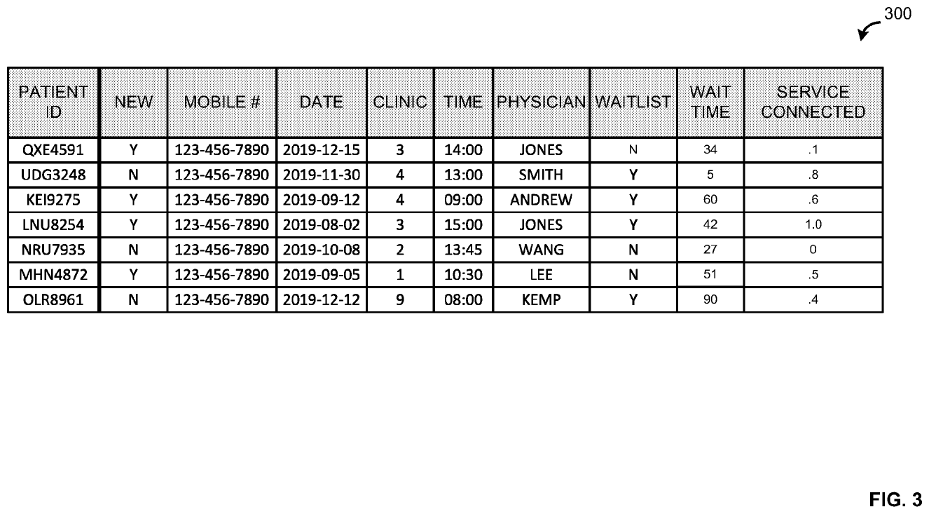

FIG. 3 illustrates an example snapshot 300 from the scheduling database 206.

Scheduling database 206 may include patient information. Patient information may comprise a patient ID or other information. A patient ID may be associated with other information such as a telephone number, whether or not the patient is a new patient, the date and/or time of an appointment at a clinic, a clinic identifier such a number or character or combination thereof, the name of a physician associated with the patient ID and/or the clinic, whether or not the patient is on a waitlist, a wait time, or an indication of service connectedness or any combination thereof.

The reminder/cancellation module 202 may be configured for sending a message to, and for receiving a message from, the messaging system 108. The reminder/cancellation module 202 may be configured for accessing the scheduling database 206. The reminder/cancellation module 202 may be configured to send and receive data. For example, the reminder/cancellation module 202 may be configured to send reminder/cancellation data. The reminder/cancellation data may indicate whether a reminder message should be sent. For example, the reminder/cancellation module may determine a date and time associated with a scheduled appointment and, prior to the scheduled appointment, cause the reminder message to be sent. The reminder/cancellation module 202 may access the scheduling database and search the database for appointments. The reminder/cancellation module 202 may search for appointments that fall a specified amount of time in the future. For example, the reminder cancellation module 202 may search for appointments 2 or 7 or 14, or any other number of days, in the future. The number of days in the future for which the reminder/cancellation module 202 searches may be fixed or may be variable. The number of days may be set by the clinic. The reminder/cancellation module 202 may search the scheduling database 206 continuously, essentially continuously, or at intervals. The reminder/cancellation module 202 may identify one or more appointments. The reminder/cancellation module 202 may determine one or more patients who have appointments with a given clinic on a specific date as indicated in the scheduling database 206. For example, the reminder/cancellation module 202 may determine one or more patients associated with the one or more appointments. The reminder/cancellation module 202 may then filter those patients on the basis of whether a cell phone number exists for a given patient in the scheduling database 206. The reminder/cancellation module 202 may construct a message to send to the patients reminding the patients of their respective appointments, along with instructions on how to cancel the appointment(s) for the date referenced in the message. The message may comprise dates, times, clinic information, facility information, and/or the like and combinations thereof. The message may comprise a template. A template may be auto-generated. A template may be stored in a database. In another embodiment, a template may be created dynamically. A template may comprise numbers, symbols, characters, or any combination thereof. For example, a template may comprise the following: “Dear Mr. [last name], an open time slot has been identified on [date] at [time] at [clinic name], located at [address]. Do you wish to accept this open time slot? Please reply Yes/No.” A patient may reply to a message. A reply may comprise an indication that the patient wishes to accept the open time slot. For example, a patient may reply, “Yes.” A reply may comprise an indication that the patient does not wish to accept the open slot. For example, a patient may reply, “No.” The message may be sent to the patient. The message may be sent by any viable communication method.

The message may comprise an interactive element and/or instructions. For example, the interactive element may be configured to receive a user input. For example, the message may comprise a hyperlink. The hyperlink may direct the user to a confirmation page or a reschedule page. The hyperlink may direct the user to a questionnaire (e.g., the health screening form). The message may comprise instructions. For example, the message may instruct the user to reply to the message to confirm or cancel the appointment. For example, the message may instruct the user to send a check-in message (e.g., the I Am Here message) when they arrive to the location of the clinic.

The reminder/cancellation module 202 may send the messages by establishing an SSL connection with the messaging system 108 (e.g., via an API) and sending the messages by making an HTTP POST to the messaging system 108 with the body of the message and the recipient phone number. The messaging system 108 may send the message to the recipient mobile device 112.

The message will be displayed on a patient's mobile device 112 via the messaging application 114. Patients can optionally reply to the message via the messaging application 114 with an indication to confirm the appointment, cancel the appointment, or to stop sending future messages. The messaging system 108 may receive the reply and send the reply to the scheduling engine 104 of the health information system 102. FIG. 4A shows an example interface 400 for the messaging application 114 resident on the mobile device 112. As shown in FIG. 4A, a message providing a reminder and an option to cancel an appointment is presented, along with a reply to cancel the appointment, and a confirmation of the cancelation.

The reminder/cancellation module 202 may then update the scheduling database 206 to indicate that the patient confirmed the appointment, or canceled the appointment. If the patient canceled the appointment, the reminder/cancellation module 202 may provide an indication to the open slot management (OSM) module 204 that a previously booked appointment is now available.

The open slot management (OSM) module 204 may be configured as a rebooking feature that may present open appointments via messages to patients. In an aspect, the OSM module 204 may access a scheduling database 206. The OSM module 204 may access the scheduling database 206 continuously or periodically or at intervals. The OSM module 204 may search the scheduling database 206 for open appointments. The OSM module 204 may search the scheduling database continuously or at intervals. The OSM module 204 may identify one or more open appointments. The OSM module 204 may add the one or more open appointments to a list. The list may be stored in a database. The OSM module 204 may access a list of eligible patients. The OSM module 204 may identify patients eligible for the one or more open appointments. The open appointments may be presented to patients. In an aspect, the open appointments may be presented to patients who have been waiting to be seen for longer than a threshold period of time (e.g., 30 days) in a clinic and offer an earlier appointment. In an aspect, the open appointments may be presented to new patients. In an additional aspect, the open appointments may be presented to patients who are associated with a service connectedness indication. In an aspect, in response to receiving an indication to cancel an appointment via the reminder/cancellation module 202, the OSM module 204 may access the scheduling database 206 to determine a patient that is on a waiting list for the same clinic as the canceled appointment (e.g., patients who have indicated that they would like to be seen at an earlier date but who are scheduled over 30 days from their desired appointment date). The OSM module 204 may construct a message to send to the patient, notifying the patient that there is an earlier appointment available, and requesting a reply regarding whether the patient would like to be rescheduled for the earlier appointment. The OSM module 204 may send the message by establishing an SSL connection with the messaging system 108 (e.g., via an API) and send the message by making an HTTP POST to the messaging system 108 with the body of the message and the recipient phone number. The messaging system 108 may send the message to the recipient mobile device 112.

The message will be displayed on a patient's mobile device 112 via the messaging application 114. The patient may optionally reply to the message via the messaging application 114 with an indication to accept the earlier appointment or to stop sending future messages. The messaging system 108 may receive the reply and send the reply to the scheduling engine 104 of the health information system 102. FIG. 4A shows an example reminder message 402 for the messaging application 114 resident on the mobile device 112. As seen in FIG. 4A, the message may comprise a reminder message. The reminder message may indicate that the patient has an upcoming appointment at a clinic. For example, the message may indicate an appointment time and an appointment date. The reminder message may indicate an appointment location (not shown) such as an address. The reminder message may comprise the interactive element and/or instructions. For example, as shown in FIG. 4A, the reminder message 402 instructs the recipient to reply to the reminder message. For example, the reminder message may instruct the recipient to reply “Y1” to confirm the appointment or “N2” to cancel the appointment. As shown in FIG. 4B, a message providing a reminder and an option to reschedule (e.g., a reschedule message) for an earlier appointment may be presented, along with a reply to accept the earlier appointment, and a confirmation of the rescheduling. For example, if the open slot management module 204 determines an open slot exists. The open slot management module may determine a patient identifier associated with the clinic who has an appointment at a time after the appointment corresponding to the open slot. Based on determining the patient identifier associated with the clinic the open slot management module may cause a message to be sent. The message may comprise a reminder message and also include an option to reschedule. The reschedule message may comprise the interactive element and/or instructions. For example, as shown in FIG. 4B, the reminder message 404 reminds the patient they have an upcoming appointment and also communicates that an earlier appointment is available. The reschedule message may instruct the recipient to interact with the interactive element and or reply in order to be rescheduled for the earlier appointment.

FIG. 4C shows an example messaging interface 406. The check-in message may be sent in response to receive the I Am Here message. The check-in message may comprise a confirmation that the recipient has arrived at the proper location. For example, a thank you with the building location may be sent. The check-in message may further comprise the interactive element and/or instructions. For example, the check-in message instructs the recipient to “please await further instruction.” Receiving the I Am Here message may cause the patient to be “checked-in” to their appointment. Causing the patient to be checked-in to their appointment may comprise sending an alert to the clinic that the patent is on site. Further, causing the patient to be checked-in to their appointment may cause relevant records to be retrieved from the database. The check-in message may comprise an indication that the patient has been checked-in and that treating medical professional is awaiting the patient's arrival. Further, the check-in message may comprise instructions. For example, the instructions may indicate a parking lot to park in, an entryway to use, an instruction to wear a mask, combinations thereof, and the like.

FIG. 4D shows another example messaging interface 408. The check-in message may be sent in response to receive the I Am Here message. The check-in message may comprise a confirmation that the recipient has arrived at the proper location. For example, a thank you with the building location may be sent. The check-in message may further comprise the interactive element and/or instructions. For example, the check-in message instructs the recipient to “please await further instruction.” Receiving the I Am Here message may cause the patient to be “checked-in” to their appointment. Causing the patient to be checked-in to their appointment may comprise sending an alert to the clinic that the patent is on site. Further, causing the patient to be checked-in to their appointment may cause relevant records to be retrieved from the database. The check-in message may comprise an indication that the patient has been checked-in and that treating medical professional is awaiting the patient's arrival. Further, the check-in message may comprise instructions. For example, the instructions may indicate a parking lot to park in, an entryway to use, an instruction to wear a mask, combinations thereof, and the like. The check-in message may comprise one or more questions. For example, the check-in message asks the recipient whether the recipient has a fever. The recipient may be prompted to select the interactive element and/or reply to the check-in message. Upon receiving the reply (e.g., confirming the patient is asymptomatic), the patient may be checked in.

The OSM module 204 may then update the scheduling database 206 to indicate that the patient confirmed the earlier appointment and canceled the later appointment.

The OSM module 204 may access electronic medical records. Electronic medical records may comprise patient information, scheduling information, clinic information, combinations thereof and the like.

The check-in module 208 may receive the I Am Here message from a patient via the module device 112 and send the check-in message in response. The check-in message may indicate that the patient is in the vicinity of the clinic at the time of the appointment (e.g., in the parking lot). Upon receiving the check-in message, the check-in module may update the database to indicate the patient has arrive. In response, the health information system may check the patient in. For example, the health information system may query the database for any records associated with the patient. For example, if the patient checks in to an eye appointment, the health information system, based on the check-in message, may query and receive any and all records associated with the patient pertaining to previous visits to the eye clinic. In response to the check-in message, the messaging system 108 may send a check-in instructional message. The check-in instructional message may comprise an interactive element and/or instructions. For example, the instructional message may instruct the patient to park in a certain parking lot, to enter a building via a certain entrance, to wear a mask inside a building, to complete a questionnaire (e.g., a health screening form), combinations thereof, and the like. A user may, based on the instructional message, complete the questionnaire, thereby generating a health screening message which may be sent to the health information system 102. For example, the interactive element may be configured to receive a user input. For example, the message may comprise a hyperlink. The hyperlink may direct the user to a confirmation page or a reschedule page. The hyperlink may direct the user to a questionnaire (e.g., the health screening form). The message may comprise instructions. For example, the message may instruct the user to reply to the message to confirm or cancel the appointment. For example, the message may instruct the user to send a check-in message (e.g., the I Am Here message) when they arrive to the location of the clinic.

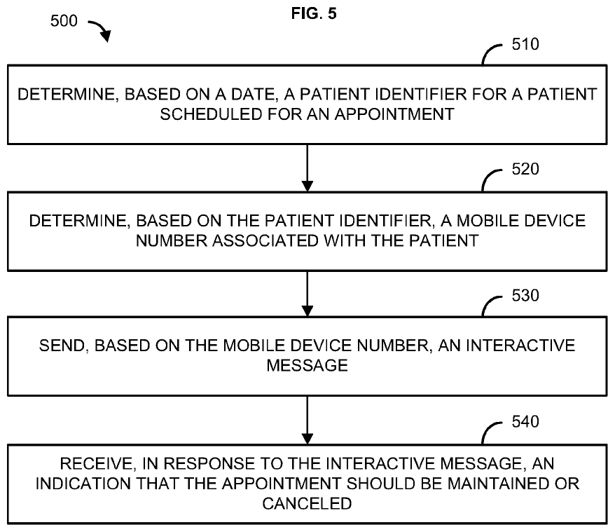

FIG. 5 shows an example method 500. The method 500 may comprise determining, based on a date, a patient identifier for a patient scheduled for an appointment at 510. Determining, based on the date, the patient identifier for the patient scheduled for the appointment may comprise determining a future date, accessing an electronic medical records database, and determining a plurality of patient identifiers associated with appointments on the future date. Determining, based on the patient identifier, a mobile device number associated with the patient may comprise determining a plurality of mobile device numbers associated with the plurality of patient identifiers.

The method 500 may comprise determining, based on the patient identifier, a mobile device number associated with the patient at 520. Determining a mobile device number comprise querying a database. For example, the database may comprise an electronic medical record (EMR) database that contains medical information and demographic information (including contact information) associated with the patient identifier.

The method 500 may comprise sending, based on the mobile device number, an interactive message at 530. The interactive message may comprise a short message service (SMS) message. Sending, based on the mobile device number, the interactive message may comprise accessing, via an application programming interface, a message server, providing message text and the mobile device number to the message server, and causing the message server to send the message text addressed to the mobile device number.

The method 500 may comprise receiving, in response to the interactive message, an indication that the appointment should be maintained or canceled at 540. Receiving, in response to the interactive message, the indication that the appointment should be maintained or canceled may comprise receiving the indication that the appointment should be canceled.

The method 500 may further comprise receiving, via an application programming interface, a cancellation signal from a message server, accessing an electronic medical records database, determining, based on the cancellation signal, the patient identifier and an appointment identifier, and canceling, based on the patient identifier and the appointment identifier, the appointment in the electronic medical records database.

The method 500 may further comprise determining, based on the appointment, an associated clinic, determining, based on the associated clinic and the date, a second patient identifier in a waiting list for an appointment with the clinic, wherein the second patient identifier has been in the waiting list for greater than a threshold period of time, determining, based on the second patient identifier, a second mobile device number, sending, based on the second mobile device number, a second interactive message offering an new appointment at the associated clinic on the date of the canceled appointment, and receiving, in response to the second interactive message, an indication that the new appointment should be established.

The method 500 may further comprise receiving, via an application programming interface, an establishment signal from the message server, accessing an electronic medical records database, determining, based on the establishment signal, the patient identifier and an appointment identifier, and establishing the new appointment in the electronic medical records database.

The method 500 may further comprise receiving an I Am Here message. The I Am Here message may indicate the patient associated with the scheduled appointment is in the vicinity of the location at which the appointment is to take place (e.g., a clinic associated with the appointment).

The method 500 may further comprising sending a check-in message. For example, in response to receiving the I Am Here message, the messaging system may cause the check-in message to be sent. The check-in message may comprise an indication that the patient has been checked-in and that treating medical professional is awaiting the patient's arrival. Further, the check-in message may comprise instructions. For example, the instructions may indicate a parking lot to park in, an entryway to use, an instruction to wear a mask, combinations thereof, and the like.

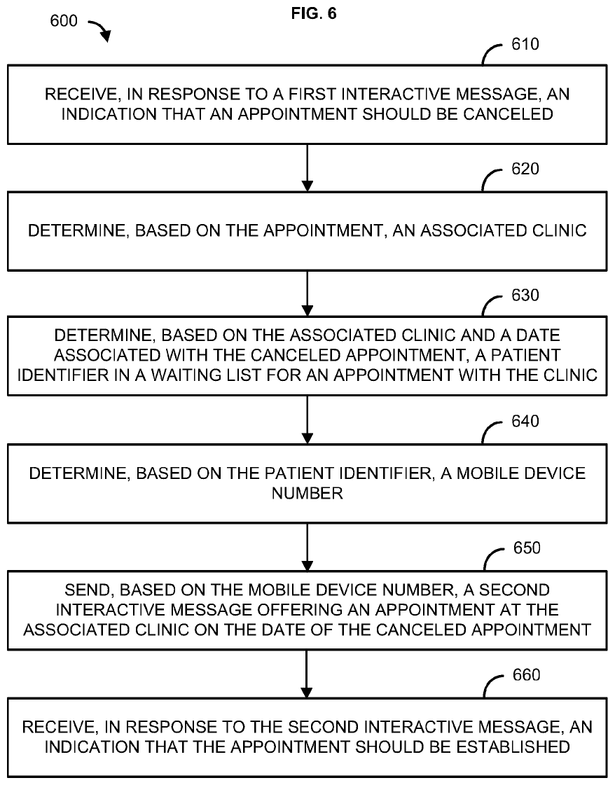

FIG. 6 shows an example method 600. The method 600 may comprise receiving, in response to a first interactive message, an indication that an appointment should be canceled at 610. The first interactive message may comprise a short message service (SMS) message.

The method 600 may comprise determining, based on the appointment, an associated clinic at 620. Determining, based on the appointment, an associated clinic may comprise determining an appointment identifier associated with the appointment and determining a clinic identifier associated with the appointment identifier.

The method 600 may comprise determining, based on the associated clinic and a date associated with the canceled appointment, a patient identifier in a waiting list for an appointment with the clinic at 630. The patient identifier may be associated with a patient who has been in the waiting list for greater than a threshold period of time.

The method 600 may comprise determining, based on the patient identifier, a mobile device number at 640. Determining a mobile device number comprise querying a database. For example, the database may comprise an electronic medical record (EMR) database that contains medical information and demographic information (including contact information) associated with the patient identifier.

The method 600 may comprise sending, based on the mobile device number, a second interactive message offering an appointment at the associated clinic on the date of the canceled appointment at 650. The second interactive message may comprise a short message service (SMS) message. The second interactive message may be associated with a timer. Sending, based on the mobile device number, the second interactive message offering the appointment at the associated clinic on the date of the canceled appointment may comprise accessing, via an application programming interface, a message server, providing message text and the mobile device number to the message server, and causing the message server to send the message text addressed to the mobile device number.

The method 600 may comprise receiving, in response to the second interactive message, an indication that the appointment should be established at 660.

The method 600 may further comprise receiving, via an application programming interface, a cancellation signal from a message server, accessing an electronic medical records database, determining, based on the cancellation signal, a patient identifier and an appointment identifier, and canceling, based on the patient identifier and the appointment identifier, the appointment in the electronic medical records database.

The method 600 may further comprise receiving, via an application programming interface, an establishment signal from the message server, accessing an electronic medical records database, determining, based on the establishment signal, the patient identifier and an appointment identifier, and establishing the new appointment in the electronic medical records database.

If a response is not received to the second interactive message within the timer, the method may comprise rescinding the offering of the appointment, determining, based on the associated clinic and the date associated with the canceled appointment, a second patient identifier in the waiting list for an appointment with the clinic, wherein the patient identifier has been in the waiting list for greater than a threshold period of time, and determining, based on the second patient identifier, a second mobile device number, and sending, based on the second mobile device number, a third interactive message offering an appointment at the associated clinic on the date of the canceled appointment.

Determining, based on the associated clinic and the date associated with the canceled appointment, the patient identifier in the waiting list for an appointment with the clinic, wherein the patient identifier has been in the waiting list for greater than a threshold period of time may comprise determining a plurality of patient identifiers, wherein the plurality of patient identifiers has been in the waiting list for greater than a threshold period of time. Determining, based on the patient identifier, a mobile device number may comprise determining, based on the plurality of patient identifiers, a plurality of mobile device numbers. Sending, based on the mobile device number, the second interactive message offering an appointment at the associated clinic on the date of the canceled appointment may comprise sending, based on the plurality of mobile device numbers, a plurality of interactive messages offering the appointment at the associated clinic on the date of the canceled appointment, the first indication that the appointment should be established will receive the appointment.

The method 600 may further comprise receiving an I Am Here message. The I Am Here message may indicate the patient associated with the scheduled appointment is in the vicinity of the location at which the appointment is to take place (e.g., a clinic associated with the appointment).

The method 600 may further comprising sending a check-in message. For example, in response to receiving the I Am Here message, the messaging system may cause the check-in message to be sent. The check-in message may comprise an indication that the patient has been checked-in and that treating medical professional is awaiting the patient's arrival. Further, the check-in message may comprise instructions. For example, the instructions may indicate a parking lot to park in, an entryway to use, an instruction to wear a mask, combinations thereof, and the like.

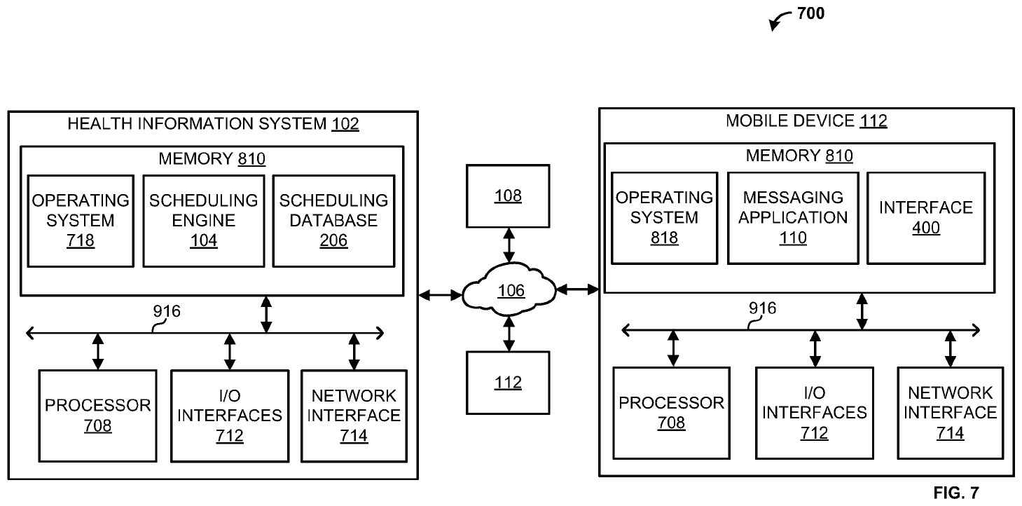

FIG. 7 is a block diagram depicting an environment 700 comprising non-limiting examples of the health information system 102 and the mobile device 112 connected through the network 106. In an aspect, some or all steps of any described method may be performed on a computing device as described herein. The health information system 102 can comprise one or multiple computers configured to store one or more of the scheduling engine 104, the scheduling database 206, and the like. The mobile device 112 can comprise one or multiple computers configured to store one or more of the messaging application 114 and to operate the user interface 400 such as, for example, a mobile phone, a tablet computer, a laptop computer, or a desktop computer. Multiple mobile devices 112 can communicate with the health information system 102 via the messaging system 108 through the network 106.

The health information system 102 and the mobile device 112 can be a digital computer that, in terms of hardware architecture, generally includes a processor 708, memory system 710, input/output (I/O) interfaces 712, and network interfaces 714. These components (708, 710, 712, and 714) are communicatively coupled via a local interface 716. The local interface 716 can be, for example, but not limited to, one or more buses or other wired or wireless connections, as is known in the art. The local interface 716 can have additional elements, which are omitted for simplicity, such as controllers, buffers (caches), drivers, repeaters, and receivers, to enable communications. Further, the local interface may include address, control, and/or data connections to enable appropriate communications among the aforementioned components.

The processor 708 can be a hardware device for executing software, particularly that stored in memory system 710. The processor 708 can be any custom made or commercially available processor, a central processing unit (CPU), an auxiliary processor among several processors associated with the health information system 102 and the mobile device 112, a semiconductor-based microprocessor (in the form of a microchip or chip set), or generally any device for executing software instructions. When the health information system 102 and the mobile device 112 is in operation, the processor 708 can be configured to execute software stored within the memory system 710, to communicate data to and from the memory system 710, and to generally control operations of the health information system 102 and the mobile device 112 pursuant to the software.

The I/O interfaces 712 can be used to receive user input from, and/or for providing system output to, one or more devices or components. User input can be provided via, for example, a keyboard and/or a mouse. System output can be provided via a display device and a printer (not shown). I/O interfaces 712 can include, for example, a serial port, a parallel port, a Small Computer System Interface (SCSI), an infrared (IR) interface, a radio frequency (RF) interface, and/or a universal serial bus (USB) interface.

The network interface 714 can be used to transmit and receive from the health information system 102, the messaging system 108, the mobile device 112 on the network 106. The network interface 714 may include, for example, a 10BaseT Ethernet Adaptor, a 100BaseT Ethernet Adaptor, a LAN PHY Ethernet Adaptor, a Token Ring Adaptor, a wireless network adapter (e.g., WiFi, cellular, satellite), or any other suitable network interface device. The network interface 714 may include address, control, and/or data connections to enable appropriate communications on the network 106.

The memory system 710 can include any one or combination of volatile memory elements (e.g., random access memory (RAM, such as DRAM, SRAM, SDRAM, etc.)) and nonvolatile memory elements (e.g., ROM, hard drive, tape, CDROM, DVDROM, etc.). Moreover, the memory system 710 may incorporate electronic, magnetic, optical, and/or other types of storage media. Note that the memory system 710 can have a distributed architecture, where various components are situated remote from one another, but can be accessed by the processor 708.

The software in memory system 710 may include one or more software programs, each of which comprises an ordered listing of executable instructions for implementing logical functions. In the example of FIG. 7, the software in the memory system 710 of the health information system 102 can comprise the scheduling engine 104 (or subcomponents thereof), the scheduling database 206, and a suitable operating system (O/S) 718. In the example of FIG. 7, the software in the memory system 710 of the mobile device 112 can comprise the user interface 400, the messaging application 114, and a suitable operating system (O/S) 718. The operating system 718 essentially controls the execution of other computer programs and provides scheduling, input-output control, file and data management, memory management, and communication control and related services.

For purposes of illustration, application programs and other executable program components such as the operating system 718 are illustrated herein as discrete blocks, although it is recognized that such programs and components can reside at various times in different storage components of the health information system 102 and/or the mobile device 112. An implementation of the scheduling engine 104, the messaging application 114, and/or the user interface 400 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method comprising:
determining, based on a date, a patient identifier for a patient scheduled for an appointment;
determining, based on the patient identifier, a mobile device number associated with the patient;
sending, based on the mobile device number, an interactive message; and
receiving, in response to the interactive message, an indication that the appointment should be maintained or canceled.

2. The method of claim 1, wherein determining, based on the date, the patient identifier for the patient scheduled for the appointment comprises:
determining a future date;
accessing an electronic medical records database; and
determining a plurality of patient identifiers associated with appointments on the future date.

3. The method of claim 2, wherein determining, based on the patient identifier, a mobile device number associated with the patient comprises determining a plurality of mobile device numbers associated with the plurality of patient identifiers.

4. The method of claim 1, wherein sending, based on the mobile device number, the interactive message comprises:
accessing, via an application programming interface, a message server;
providing message text and the mobile device number to the message server; and
causing the message server to send the message text addressed to the mobile device number.

5. The method of claim 1, further comprising:
receiving, from a mobile device, an indication that a patient associated with the patient identifier has arrived;
determining, based on the mobile device number associated with the patient, the appointment
sending, based on the indication that the patent has arrived and the appointment, an instructional message wherein the instructional message comprises one or more of an interactive element and instructions;
receiving, based at least one of the interactive element or instructions, health information associated with the patient; and
updating an appointment status.

6. The method of claim 1, wherein receiving, in response to the interactive message, the indication that the appointment should be maintained or canceled comprises receiving the indication that the appointment should be canceled.

7. The method of claim 6, further comprising:
receiving, via an application programming interface, a cancellation signal from a message server;
accessing an electronic medical records database;
determining, based on the cancellation signal, the patient identifier and an appointment identifier; and
canceling, based on the patient identifier and the appointment identifier, the appointment in the electronic medical records database.

8. The method of claim 6, further comprising:
determining, based on the appointment, an associated clinic;
determining, based on the associated clinic and the date, a second patient identifier in a waiting list for an appointment with the associated clinic, wherein the second patient identifier has been in the waiting list for greater than a threshold period of time;
determining, based on the second patient identifier, a second mobile device number;
sending, based on the second mobile device number, a second interactive message offering a new appointment at the associated clinic on the date of the canceled appointment; and
receiving, in response to the second interactive message, an indication that the new appointment should be established.

9. The method of claim 8, further comprising:
receiving, via an application programming interface, an establishment signal from the message server;
accessing an electronic medical records database;
determining, based on the establishment signal, the patient identifier and an appointment identifier; and
establishing the new appointment in the electronic medical records database.

10. A method comprising:
receiving, in response to a first interactive message, an indication that an appointment should be canceled;
determining, based on the appointment, an associated clinic;
determining, based on the associated clinic and a date associated with the canceled appointment, a patient identifier in a waiting list for an appointment with the associated clinic, wherein the patient identifier has been in the waiting list for greater than a threshold period of time;
determining, based on the patient identifier, a mobile device number;
sending, based on the mobile device number, a second interactive message offering an appointment at the associated clinic on the date of the canceled appointment; and
receiving, in response to the second interactive message, an indication that the appointment should be established.

11. The method of claim 10, wherein the first interactive message comprises a short message service (SMS) message and wherein the second interactive message comprises a short message service (SMS) message.

12. The method of claim 10, further comprising:
receiving, via an application programming interface, a cancellation signal from a message server;
accessing an electronic medical records database;
determining, based on the cancellation signal, a patient identifier and an appointment identifier; and
canceling, based on the patient identifier and the appointment identifier, the appointment in the electronic medical records database.

13. The method of claim 10, wherein determining, based on the appointment, an associated clinic comprises determining an appointment identifier associated with the appointment and determining a clinic identifier associated with the appointment identifier.

14. The method of claim 10, wherein sending, based on the mobile device number, the second interactive message offering the appointment at the associated clinic on the date of the canceled appointment comprises:
accessing, via an application programming interface, a message server;
providing message text and the mobile device number to the message server; and
causing the message server to send the message text addressed to the mobile device number.

15. The method of claim 10, further comprising:
receiving, via an application programming interface, an establishment signal from the message server;
accessing an electronic medical records database;
determining, based on the establishment signal, the patient identifier and an appointment identifier; and
establishing a new appointment in the electronic medical records database.

16. The method of claim 10, wherein the second interactive message is associated with a timer.

17. The method of claim 16, wherein if a response is not received to the second interactive message within the timer, the method comprises:
rescinding the offering of the appointment;
determining, based on the associated clinic and the date associated with the canceled appointment, a second patient identifier in the waiting list for an appointment with the associated clinic, wherein the patient identifier has been in the waiting list for greater than a threshold period of time;
determining, based on the second patient identifier, a second mobile device number; and
sending, based on the second mobile device number, a third interactive message offering an appointment at the associated clinic on the date of the canceled appointment.

18. The method of claim 10, wherein determining, based on the associated clinic and the date associated with the canceled appointment, the patient identifier in the waiting list for an appointment with the associated clinic, wherein the patient identifier has been in the waiting list for greater than a threshold period of time comprises determining a plurality of patient identifiers, wherein the plurality of patient identifiers has been in the waiting list for greater than a threshold period of time.

19. The method of claim 18, wherein determining, based on the patient identifier, a mobile device number comprises determining, based on the plurality of patient identifiers, a plurality of mobile device numbers.

20. The method of claim 19, wherein sending, based on the mobile device number, the second interactive message offering an appointment at the associated clinic on the date of the canceled appointment comprises sending, based on the plurality of mobile device numbers, a plurality of interactive messages offering the appointment at the associated clinic on the date of the canceled appointment, a first indication that the appointment should be established will receive the appointment.
